(19) United States
(12) Patent Application Publication
(10) Pub. No.: US 2004/0110714 A1
(43) Pub. Date: Jun. 10, 2004

(54) Stabilized naked DNA compositions

(76) Inventors: Roger C. Adami, Noank, CT (US); Jefferson D. Knight, New Haven, CT (US); Larry A. Gatlin, Groton, CT (US)

Correspondence Address: SCULLY SCOTT MURPHY & PRESSER, PC, 400 GARDEN CITY PLAZA, GARDEN CITY, NY, 11530

(21) Appl. No.: 10/675,130

(22) Filed: Sep. 30, 2003

Related U.S. Application Data
(60) Provisional application No. 60/417,189, filed on Oct. 9, 2002.

(51) Int. Cl. A61K 48/00

(52) U.S. Cl. 514/44

(57) ABSTRACT

The present invention relates to a method to condense DNA without any high-molecular-weight condensing agents by condensing plasmid DNA with a divalent cation and a lyophilizable alcohol. The invention also relates to an aqueous composition which includes condensed plasmid DNA and a carrier such as a lyophilizable, water-miscible alcohol and a divalent cation.

### CROSS REFERENCE TO RELATED APPLICATIONS

The present application claims benefit of U.S. Provisional Application No. 60/417,189 filed Oct. 9, 2002.

### BACKGROUND OF THE INVENTION

The pharmaceutical application of naked DNA delivery systems requires stabilization of DNA during both the manufacturing process and for long-term storage. DNA is a labile molecule that is susceptible not only to enzymatic and chemical degradation via hydrolytic and oxidative pathways, but also mechanical damage, e.g., induced by shear. Processing of DNA during manufacture of a drug product can result in many medium- and high-shear processes that affect the stability of the DNA. One such process is the freezing step during lyophilization. Additionally, turbulent flow in tubing and filtration are processes that increase the shear-stress to which the DNA is subjected. Since any strand breakage that occurs in DNA affects the quality and performance of the drug product it is imperative to address the potential of shear related damage that may occur during processing of the DNA.

Protecting DNA from damage is of paramount importance in biological systems. In nature, condensation and packaging of chromosomal DNA has evolved to not only reduce the size of the DNA, but more importantly to stabilize the DNA. Although organisms have elaborate mechanisms for packaging their DNA, a similar collapse of extended DNA into compact structures is seen in vitro through the addition of various reagents. Many of these reagents, including cationic lipids (Zhang, 1997), peptides (Wyman, 1997), and cationic polymers (Ogris, 1998), have been studied as transfection agents in nonviral gene therapy. These reagents typically ion-pair with the anionic phosphate backbone of DNA resulting in the condensation of the DNA, which provides a compact form for gene delivery. Additionally, it has been shown that the polycationic condensing agents protect DNA from nucleolytic enzyme and sonication-induced degradation (Adami, 1999). Despite their many advantages, however, high-MW condensing agents are not without their drawbacks and have met limited success in vivo due to their cytotoxicity (Wolfert, 1996) and complement activation (Planck, 1996).

Recently, administration of naked DNA has been gaining acceptance as a preferred method of DNA delivery for nonviral gene therapy. The concept is simple and yet quite effective for in vivo administration of DNA. Naked DNA is typically plasmid DNA, without complexation excipients, formulated in a buffer that protects the DNA from chemical degradation, although it is also commonly lyophilized to extend room-temperature stability. Although the formulation is simple, manufacturing the final drug product requires stabilizing the labile naked DNA to the shear-stresses it may face during production.

### SUMMARY OF THE INVENTION

The present invention relates to a method to condense DNA without any high-molecular-weight condensing agents, comprising condensation of plasmid DNA with a divalent cation and a lyophilizable alcohol.

The present invention also relates to an aqueous composition comprising condensed plasmid DNA and a carrier, wherein the carrier comprises a lyophilizable, water-miscible alcohol and a divalent cation. In an embodiment of the invention, the lyophilizable water-miscible alcohol is tert-butanol. In another embodiment of the invention, the concentration of tert-butanol is from about 15% to about 35% by volume, more preferably from about 17% to about 25% by volume, and in a particularly preferred embodiment, the concentration of tert-butanol is about 20% by volume.

In another embodiment of the invention, the divalent cation is selected from the group consisting of Ca⁺², Mg⁺² or Zn⁺², preferably, the divalent cation is Ca⁺², more preferably, the divalent cation is Ca⁺² and the concentration of the Ca⁺² is from about 0.2 to about 2 millimolar. In a still more preferred embodiment, the divalent cation is Ca+² and the concentration of the Ca+² is about 1 millimolar.

In another embodiment of the invention, the divalent cation is Ca⁺² and the concentration of DNA is from about 10 ug/mL to about 200 ug/mL. In another embodiment of the invention, the molar ratio of Ca⁺² to DNA-phosphate is about 3. In yet another embodiment of the invention, the concentration of Ca+² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

In another embodiment of the invention, the counterion to the divalent cation is chloride.

In another embodiment of the invention, the DNA is stable to shear, including shear induced by sonication. In an embodiment of the invention, the plasmid DNA remains intact following sonication for 60 seconds using a 50 watt probe sonicator. In another embodiment of the invention, the total percent of supercoiled, open circular and linear plasmid DNA together after sonication is greater than 90% of its initial value.

In an embodiment of the invention, the DNA in the condensate consists essentially of toroids and rods. In another embodiment of the invention, the toroids exhibit a median particle size in the range of from about 10 to about 500, and preferably about 50 to about 100 nanometers, as measured by electron microscopy.

In an embodiment of the invention, the condensate exhibits a bimodal particle size distribution. In another embodiment of the invention, the particle size distribution of the condensate, measured by dynamic light scattering, exhibits peaks in the range of from about 40 to about 70 nanometers and from about 200 to about 500 nanometers.

This invention also relates to a method to condense plasmid DNA comprising:

(a) preparing an aqueous solution of deionized plasmid DNA;

(b) adding a lyophilizable, water-miscible alcohol to the solution of step (a); and

(c) adding a divalent cation to the mixture of step (b).

In an embodiment of the method of this invention, the lyophilizable water-miscible alcohol is tert-butanol. In another embodiment of the method of this invention, the concentration of tert-butanol is from about 15% to about 35% by volume, more preferably, from about 17% to about 25% by volume, and in a particularly preferred embodiment, the concentration of tert-butanol is about 20% by volume. In another embodiment of the method of this invention, the divalent cation is selected from the group consisting of Ca⁺², Mg⁺² or Zn⁺², preferably, the divalent cation is Ca⁺². In another embodiment of the method of this invention, the concentration of Ca⁺² is from about 0.2 to about 2 millimolar, more preferably, the concentration of Ca⁺² is about 1 millimolar, and in a particularly preferred embodiment of the method, the concentration of DNA is from about 10 ug/mL to about 200 ug/mL.

In another embodiment of the method of this invention, the molar ratio of Ca⁺² to DNA-phosphate is about 3.

In another embodiment of the method of this invention, the concentration of Ca⁺² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

In another embodiment of the method of this invention, the counterion to the divalent cation is chloride. The present invention also contemplates counterions to the divalent cation, such as, but not limited to calcium salts, including carbonate, phosphate, edate, acetate, oxalate, gluconate and lactate.

In another embodiment of the method of this invention, the method further comprises (d) removing water and the lyophilizable, water-miscible alcohol from the composition; in a preferred embodiment thereof, the water and the lyophilizable, water-miscible alcohol are removed by lyophilization. In another embodiment of the method of the invention, step (d) comprises spray-drying the composition.

In another embodiment, the present invention contemplates a method of making a DNA condensate including toroids, rods and spheres comprising:

(a) preparing an aqueous solution of deionized plasmid DNA;

(b) adding a lyophilizable, water-miscible alcohol to the solution of step (a); and

(c) adding a divalent cation to the mixture of step (b).

A composition comprising condensed plasmid DNA and a divalent cation wherein said composition is substantially free of stabilizing excipients after removal of solvent system via spray drying, lyophilization, or evaporation.

### DESCRIPTION OF THE DRAWINGS

FIG. 1: Conditions for condensation of DNA in tbuOH and CaCl₂. Condensation was determined by centrifugation and absorbance and/or by count rate comparison of particle size measurements.

FIG. 2: Calcium chloride induced condensation leads to compaction of the DNA which can be quantified using quasielastic light scattering particle sizing.

FIG. 3: Transmission electron microscope photos of DNA in 20% tert-butanol with 1 mM CaCl₂ (panel B) and without 1 mM CaCl₂ (panel A). The magnification of both photos is (50,000×).

FIG. 4: Temperature dependence of particle formation.

FIG. 5: Kinetics of Particle Formation.

FIG. 6: Shear protection of DNA in condensed (a) and uncondensed (b) form.

### DETAILED DESCRIPTION OF THE INVENTION

As described above, administration of naked DNA has been gaining acceptance as a preferred method of DNA delivery for nonviral gene therapy. Naked DNA is typically plasmid DNA, without complexation excipients, formulated in a buffer that protects the DNA from chemical degradation, although it is also commonly lyophilized to extend room-temperature stability. Although the formulation is simple, manufacturing the final drug product requires stabilizing the labile naked DNA to the shear-stresses it may face during production.

Applicants have addressed the need for a stable formulation of naked DNA by condensing plasmid DNA, via treatment with calcium chloride in 20% (v/v) tert-butanol, so as to form small (˜50 nm) toroids as well as larger (˜300 nm) rods and spheres i.e. condensed particles of DNA. The condensed particles retained a negative surface charge, indicating sub-stoichiometric concentrations of calcium. As demonstrated below, applicants' DNA compositions provide a greater than tenfold protection against sonication-induced shear stress.

A preferred embodiment of the invention is used to prepare stable formulations of DNA suitable for further processing. In this embodiment, purified deionized plasmid DNA at a concentration of 0.1 μg/mL to 1 mg/mL, more preferably from 1 μg/mL to 500 μg/mL, still more preferably at 10 μg/mL to 200 μg/mL and most preferred at 100 μg/mL is dissolved in an aqueous solution of t-butanol ranging in concentration from 17% to 25% (v/v). An appropriate divalent cation consisting of Ca⁺², Mg⁺², or Zn²⁺ at concentrations of 0.2 mM to 2 mM would then be added to the t-butanol cosolvent solution to condense the DNA. The stoichiometric ratio of anionic phosphates of the DNA backbone to divalent cations is preferred to be between (anions/cations) 0.1 and 1.0, with approximately 0.3 being the most preferred ratio. The solution is then allowed to equilibrate, for approximately 45 min. to permit thermodynamic equilibrium condensation to be attained. The rods, toroids, and spherical DNA particulates will fall in the size range of approximately 20-500 nm. The solution containing the condensed DNA is then transferred to downstream process unit operations, such as sterile filtration through 0.22 um filters, spray-drying, or lyophilization.

This solution is manufactured in large-scale commercial processing equipment, such as 300 gallon stainless steel compounding tanks.

The sterile filtered condensed DNA is then be processed by lyophilization or spray-drying to obtain stable pharmaceutical dosage forms. Prior to lyophilization the DNA can be combined with bulking agents such as sucrose, mannitol, trehalose, lactose, or other common bulking agents. The t-butanol solution freezes and forms a single phase amenable to lyophilization due to the sublimation properties of t-butanol. In the dried lyophilized cake the DNA toroids and rods remain intact. Upon reconstitution the lyophile cake would rapidly dissolve and the DNA would be completely solubilized into uncondensed native plasmid DNA ready for dosing with the only traces of the original process being the cations and any added bulking agents. It is substantially free of t-butanol at this stage of the process.

An alternate approach to processing the DNA is to utilize spray drying. Spray drying involves three fundamental unit processes: liquid atomization, gas-droplet mixing, and drying from liquid droplets. Atomization is accomplished usually by one of three atomizing devices: high-pressure nozzles, two-fluid nozzles, and high-speed centrifugal disks. With these atomizers, thin solutions may be dispersed into droplets as small as 2 μm. The largest drop sizes rarely exceed 500 μm (35 mesh). Because of the large total drying surface and small droplet sizes created, the actual drying time in a spray dryer is typically not more than about 30 seconds.

One of the principal advantages of spray drying is the production of a spherical particle, which is usually not obtainable by any other drying method. The spherical particle may be solid or hollow, depending on the material, the feed condition, and the drying conditions. Because of the high heat-transfer rates to the drops, the liquid at the center of the particle vaporizes, causing the outer shell to expand and form a hollow sphere.

The dried DNA particles can be used as a powdered form of the DNA ready to be reconstituted into a hydrating solution for parenteral administration including, but not limited to intraveneous, intramuscular and intraperitoneal administration. The reconstituted DNA of the present invention can also be administered subcutaneously and intraocularly. The reconstituted DNA can also be administered by aerosol means and can be delivered in a dried particulate form directly in a powder by aerosol or other inhalatory administration means. The composition of powdered DNA for dosing is substantially free of the solvents used to modify its structure.

This invention also relates to a composition prepared by any of the above methods. This invention also contemplates stable formations of RNA prepared in accordance with the methods for preparing stable formulations of DNA. Thus, the present application contemplates preparations of condensed naked-RNA formulations suitable for RNA delivery therapeutic application, including but not limited to non-viral gene therapy.

In this application, the term “carrier” means any inactive ingredient, i.e., other than plasmid DNA. It is contemplated that carriers in addition to the lyophilizable alcohol and divalent cation may be added to the composition of the invention for use in further processing of the aqueous composition or, preferably, a lyophilized composition of the invention. Carriers useful for the preparation of pharmaceutical compositions are well-known in the art (see Remington: The Practice of Pharmacy, Lippincott Williams and Wilkin,. Baltimore Md., 20^{th}ed. 2000). It is within the routine practice of the art-skilled to select and determine which carriers are appropriate for the intended application of the compositions of the invention. Some examples of carriers include inert diluents or fillers, binders and excipients, including ingredients useful for enhancing palatability, e.g., flavorings. Other pharmaceutically acceptable carriers useful in preparing compositions for oral administration, e.g., tablets, include disintegrants such as starch, alginic acid and certain complex silicates and with binding agents such as sucrose, gelatin and acacia. Additional pharmaceutically acceptable carriers include lubricating agents such as magnesium stearate, sodium lauryl sulfate and talc are often useful for tableting purposes. Solid compositions of a similar type may also be employed in soft and hard filled gelatin capsules; preferred materials therefore include the pharmaceutically acceptable carriers lactose, milk sugar and high molecular weight polyethylene glycols.

### EXAMPLES

In the following examples the following materials and plasmid DNA were used. Plasmid DNA encoding feline erythropoietin (80% supercoiled, 18% open circular), was used in the experiments below. Construction, sequence and expression of the plasmid is described in European Patent Application 99 309201.4, published on Jun. 28, 2000 as EP 1 013 288 A2, the contents of which are hereby incorporated by reference in their entirety.

The salts, zinc chloride and magnesium chloride hexahydrate, were from Aldrich, and the calcium chloride was from Fisher. The alcohols used were Methanol (J. T. Baker), 2-methyl-2-propanol (tert-butanol, tbuOH) (Aldrich), and ethanol (Pharmco). All dilutions were into water purified via the Alpha-Q water purification system (Millipore). Stocks of DNA, alcohol, salt, and water were prepared and filtered through a Millex-GP 0.22 μm filter unit prior to use.

### Example 1

### Preparation of Condensed Naked-DNA Formulations

A deionized solution of plasmid DNA (5600 BP) was prepared by washing the DNA on a 10,000 MWCO Amicon ultra-dialysis membrane using 10 volumes of deionized water. The deionized DNA was dissolved in diH₂O to a 1 mg/ml stock solution. The DNA was diluted to the desired concentration in an aqueous alcohol solution. The alcohols used were methanol, ethanol, isopropanol, or tert-butanol. Various amounts of the calcium salt form of zinc, magnesium, or calcium were added to the alcohol/DNA solution. The solutions were mixed well by vortexing and incubated for 1-1.5 hr at room temperature.

### Example 2

### Centrifugation-absorbance Assays

Condensation of DNA was determined by centrifugation and absorbance measurement. Samples were centrifuged 4 min at 15,800 g. An 80-μL aliquot from the top of the supernatant was diluted 1:10 and concentration of DNA was measured (A₂₆₀ nm) and compared to that of a corresponding aliquot taken prior to centrifugation. Alternatively, for sample DNA concentrations ≦50 μg/mL, the aliquots were diluted into 1×GelStar DNA stain (FMC BioProducts) and analyzed on a Hitachi F-2000 fluorescence spectrophotometer (excitation 493 nm, emission 527 nm) with similar results.

Identification of formulations that led to DNA condensation was performed by a centrifugation assay. Condensed or aggregated samples showed a 5-10 fold decrease in absorbance after centrifugation, while uncondensed samples had a reduction of 15% or less. DNA (100 μg/mL) in various concentrations of MgCl₂, CaCl₂, and ZnCl₂ and methanol, ethanol, or tert-butanol were investigated. Conditions selected to perform the condensation were 20% tert-butanol and 1 mM CaCl₂. In all solvents except tert-butanol, aggregation (formation of visible flocculates) occurred most rapidly with MgCl₂. However, the magnesium salt was the least effective of the three salts for condensing DNA in t-buOH solutions, requiring a higher concentration to induce condensation. In 80% (v/v) t-buOH, the solubility of DNA was exceeded and DNA precipitation occurred overnight in the presence of any ion. A factorial design experimental methodology was used to determine the optimal conditions that led to condensation (data not shown). Conditions that led to robust DNA condensation occurred at 100 μg/mL plasmid DNA in 20% tert-butanol with 1 mM CaCl₂.

The results of the initial salt-alcohol condensation screening assay are shown in Table 1. Samples were observed following the addition of the indicated amount of salt to solvent containing 100 μg/mL plasmid DNA. The numbers indicate time (hrs) when visible aggregation was first noticed. A dash indicates samples that did not show a decrease in absorbance after centrifugation. The sample point that was condensed but did not visibly aggregate is marked “condensed”.

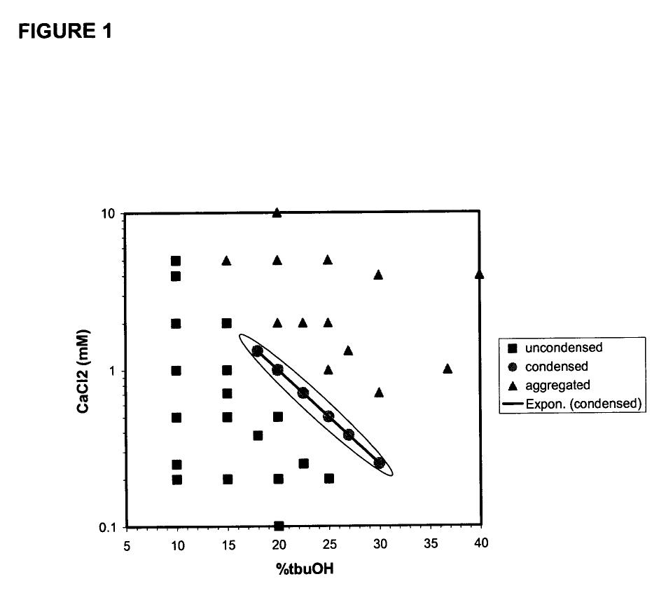

Optimization of conditions leading to condensation without aggregation in 20% tert-butanol and 1 mM CaCl₂ was performed by varying solution conditions around this point (FIG. 1). The equation of the line of fit is [salt]=16 exp(0.1386*%t), where [salt] is the millimolar CaCl₂ concentration and %t is percent tert-butanol. Condensation without aggregation was found in a range of conditions, with less CaCl₂ required as the tert-butanol concentration increased. The effect of varying the DNA concentration was also investigated, and it was found that more CaCl₂ was required for condensation as the DNA concentration was increased, although this dependence was much less dramatic than that of the salt-alcohol concentrations.

TABLE 1

| | 50% | | 20% tert- | 50% tert- | 80% tert- |
|---|---|---|---|---|---|
| Salt | Methanol | 50% Ethanol | butanol | butanol | butanol |
| 0.1 mM | — | — | — | — | 24 |
| CaCl₂ | | | | | |
| 1 mM | 4 | 3 | Condensed | 24 | 24 |
| CaCl₂ | | | | | |
| 10 mM | 1.5 | 3 | 3 | 24 | 1 |
| CaCl₂ | | | | | |
| 0.1 mM | — | — | — | — | 24 |
| ZnCl₂ | | | | | |
| 1 mM | 3 | 3 | — | 24 | 24 |
| CaCl₂ | | | | | |
| 10 mM | 4 | 3 | 3 | 24 | 24 |
| ZnCl₂ | | | | | |
| 0.1 mM | — | — | — | — | 24 |
| MgCl₂ | | | | | |
| 1 mM | 3 | 3 | — | — | 24 |
| MgCl₂ | | | | | |
| 10 mM | 1 | 1 | 3 | 2 | 1 |
| MgCl₂ | | | | | |

### Example 3

### Particle Size Measurements

Particle size measurements were performed on a 90Plus Particle Size Analyzer from Brookhaven Instruments Corporation, Holtsville, N.Y. Three runs of 1 min each were performed on each sample, and the mean diameter of the three runs was reported. Solvent viscosity of a 20% tert-butanol solution was 1.723 cP as measured on a TA Instruments AR1000 rheometer and was used to measure particle size in the dynamic light scattering mode.

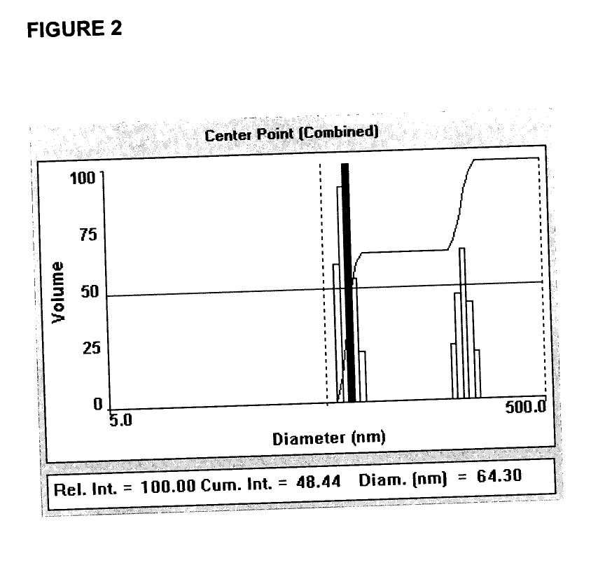

Particle size data for DNA condensed with 20% tert-butanol and 1 mM CaCl₂ indicated that two populations of particles spontaneously formed in solution. The diameters of the two populations of particles were 40-70 nm and 200-500 nm. These sizes correspond to two forms seen with electron microscopy: a toroid form with diameter of about 50-100 nm and a rodlike form about 50 nm in width and several hundred nm in length (FIG. 2). In this figure there is a bimodal distribution of particle sizes due to differences in the hydrodynamic radii of the smaller diameter toroids and larger diameter rods following CaCl₂ condensation. In this sample 63% of the particles (by volume) have a diameter centered at 64 nm and 37% of the particles have a diameter centered at 220 nm.

### Example 4

### Electron Microscopy

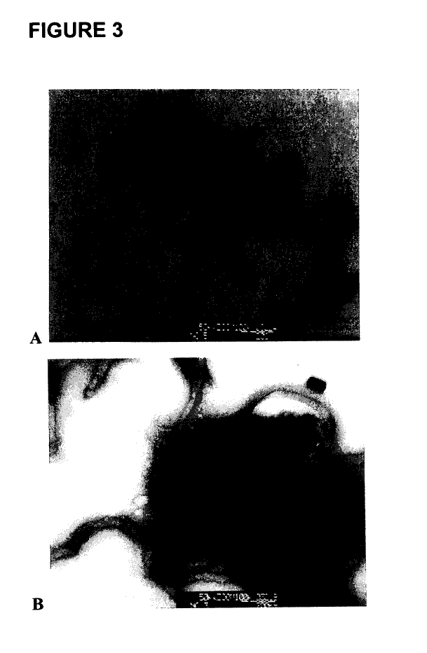

Samples were prepared and incubated at room temperature for 1.5 hrs before being stained for electron microscopy. Formvar coated copper grids (200 mesh) were treated by glow discharge for 2 min. A drop of the DNA was floated on the grid for 60 sec. and blotted dry. A 2% uranyl acetate stain was then applied for 60 sec. and blotted dry. Grids were viewed on a Hitachi electron microscope at 50,000× magnification at 100 kV power. The magnification of both photos is (50,000×). FIG. 3 shows the plasmid DNA structures obtained in 20% tert-butanol with 1 mM CaCl₂ (panel B) and without 1 mM CaCl₂ (panel A) Panel B shows the presence of rods and toroids (˜100 nm diameter) of DNA following condensation with 1 mM CaCl₂.

### Example 5

### Kinetics of Particle Formation

The kinetics of particle formation were investigated with a Hitachi F-2000 fluorescence spectrometer with excitation and emission wavelengths set to 400 nm. The total light scattered of an equilibrated solution of DNA in tert-butanol at 90° was measured for a 5 sec interval. Increase in light scattered was measured as a function of time after the addition and thorough mixing of CaCl₂ to the DNA/tert-butanol solution.

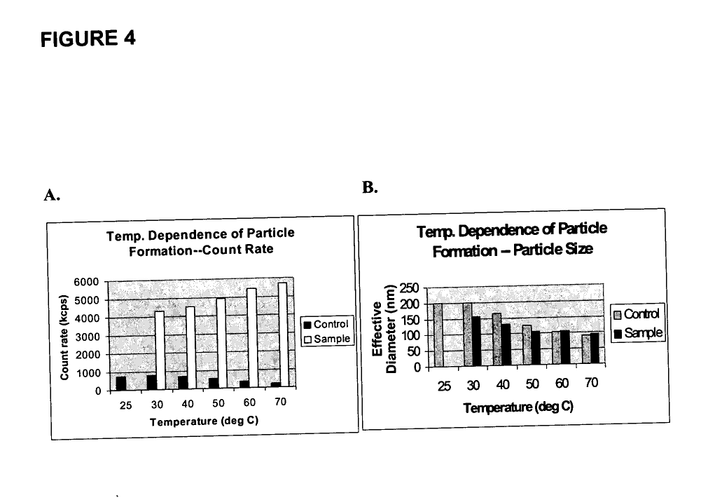

The effect of temperature on particle formation relative to background was studied using particle size measurements (FIG. 4). As shown in FIG. 4a, the count rate increased with increasing temperature while the background count rate decreased with increasing temperature. The reported particle size decreased for both sample and control with increasing temperature, although the decrease between 30° and 70° was greater for the control (55%) than the DNA-containing sample (40%) (FIG. 4b). Monitoring condensation upon addition of CaCl₂ yielded fully condensed DNA as demonstrated in the plateau region of the figure, which corresponds to maximum light scattering intensity. The 400 nm particle size was used to monitor condensation and occurred 45 minutes after addition of calcium chloride. The control sample of 20% t-buOH demonstrates a scatter intensity less than one order of magnitude than that of the condensed particle.

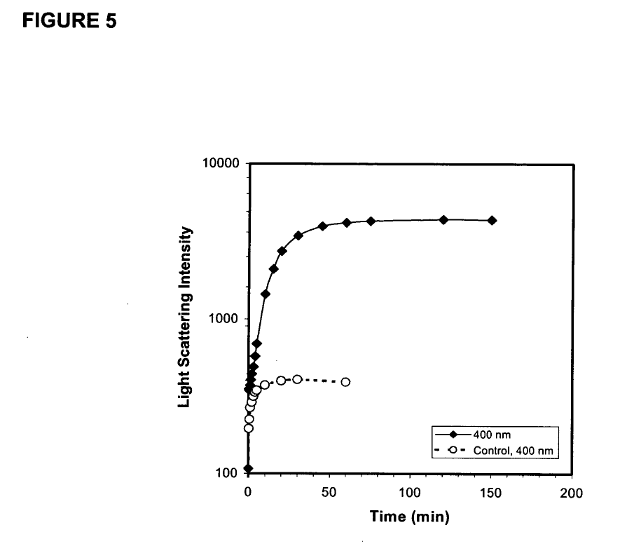

The kinetics of particle formation was investigated using total intensity light scattering at 400 nm (FIG. 5). The results show two phases of particle formation: the first, seen also in the control, occurs within 2 min after the addition of CaCl₂ and is thought to reflect a structural rearrangement of the solvent system upon the addition of salt. After 5 min, the light scattering intensity is dominated by condensing DNA, which reaches a plateau after 1 hr and remains there for several hours. Visible aggregation of these particles was not generally noticed after 24 hr, although the fraction of particles in the 200-500 nm size range was greater.

### Example 6

### Measurement of Zeta Potential

The zeta potential of DNA condensed with 20% tbuOH and 1 mM CaCl₂ was measured using a 90Plus Particle Size Analyzer from Brookhaven Instruments Corporation (Holtsville, N.Y.). Zeta potential was calculated using the Smoluchowski model with the viscosity set at 1.723 cP and the dielectric constant as 66.5. Six runs of 15 cycles each were performed and the average zeta measurement was reported.

The zeta potential of DNA particles condensed with 20% tbuOH and 1 mM CaCl₂ was determined to be −17.28+/−1.29 mV under an electric field of 7.24 V/cm.

### Example 7

### Shear-Stress Resistance

Samples were prepared as described above and incubated for 24 hr at room temperature. One milliliter aliquots of condensed DNA formulations and uncondensed controls were sonicated for various amounts of time using a Cole Parmer 4710 Series 50 W sonicator. Damage to the DNA was analyzed by electrophoresis through a 1.1% Seakem agarose gel (FMC BioProducts) electrophoresed for 60 min at 80 V and stained using Sybr®-Gold (Molecular Probes, Eugene Oreg.). A linear DNA marker was obtained by restriction digest of the plasmid DNA with EcoRV (Gibco).

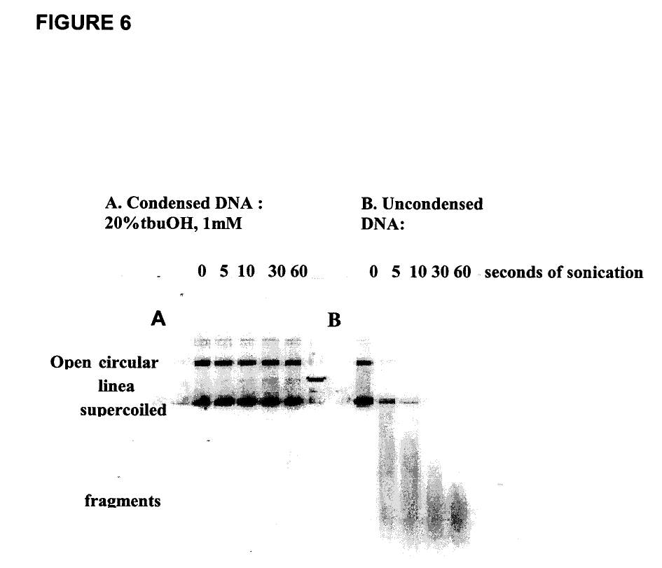

The shear stress stability of DNA condensed with 20% tbuOH and 1 mM CaCl2 was investigated via sonication. The primary structure of DNA condensed with 20% tbuOH and 1 mM CaCl₂ was found to be protected from shear stress induced via sonication (FIG. 6, quantified in Table 2 below). Numbers above the lanes indicate time (seconds) of exposure to 50 W probe sonication. Following 30 sec of sonication 100% of the uncondensed open circular, linear, and supercoiled forms of the plasmid DNA are completely degraded resulting in the fragment smear seen at the bottom of the gel. In contrast, the condensed DNA retains 100% of the supercoiled and open circular form of the plasmid DNA following 60 sec of sonication.

While uncondensed DNA is degraded into oligonucleotide fragments after as little as 5 s of sonication, the majority of the condensed DNA was still in its initial supercoiled and open circular forms after 60 s of sonication. This protection from shear stress is afforded the DNA simply through condensation without the presence of polymers or other macromolecules.

The cavitation induced shear-stress protection afforded to condensed plasmid DNA is quantified in Table 2. The top panel has sonication-induced shear data for condensed DNA. The total percent of intact DNA, defined as supercoiled, open circular, and linear forms of plasmid DNA, was 100% after 60 seconds of probe sonication (50 W). There was only a 3% loss of supercoiled DNA after 60 sec. An uncondensed control plasmid DNA is shown in the bottom panel. It is seen that more 60 percent of the DNA is degraded after only 5 sec of sonication, and only 1.6 percent of the intact plasmid DNA remains after 60 sec.

TABLE 2

| | | | | | | |
|---|---|---|---|---|---|---|
| | | | | Total | | % of Intact |
| Sample | Band | Volume | Area | Intensity | % of Total | Plasmid |

Protected plasmid

| | | | | | | |
|---|---|---|---|---|---|---|
| 0 Sec | SC | 195,663 | 462 | 293,808 | 66.6 | 100.0 |
| | Linear | 9,458 | 210 | | 3.2 | |
| | OC | 88,686 | 378 | | 30.2 | |
| 5 sec | SC | 192,927 | 462 | 292,623 | 65.9 | 99.6 |
| | Linear | 11,889 | 189 | | 4.1 | |
| | OC | 87,807 | 378 | | 30.0 | |
| 10 Sec | SC | 203,669 | 462 | 311,326 | 65.4 | 106.0 |
| | Linear | 16,964 | 210 | | 5.4 | |
| | OC | 90,694 | 378 | | 29.1 | |
| 30 sec | SC | 206,723 | 462 | 322,298 | 64.1 | 109.7 |
| | Linear | 21,862 | 210 | | 6.8 | |
| | OC | 93,713 | 378 | | 29.1 | |
| 60 sec | SC | 188,993 | 462 | 301,073 | 62.8 | 102.5 |
| | Linear | 24,913 | 210 | | 8.3 | |
| | OC | 87,167 | 378 | | 29.0 | |

Control (unprotected)

| | | | | | | |
|---|---|---|---|---|---|---|
| 0 Sec | SC | 214,926 | 462 | 308036 | 69.8 | 100 |
| | Linear | 20,770 | 210 | | 6.7 | |
| | OC | 72,340 | 378 | | 23.5 | |
| 5 sec | SC | 97,321 | 462 | 118265 | 82.3 | 38.4 |
| | Linear | 7,607 | 189 | | 6.4 | |
| | OC | 13,337 | 378 | | 11.3 | |
| 10 Sec | SC | 37,332 | 462 | 44953 | 83.0 | 14.6 |
| | Linear | 3,415 | 210 | | 7.6 | |
| | OC | 4,205 | 378 | | 9.4 | |
| 30 sec | SC | 7,672 | 462 | 10461 | 73.3 | 3.4 |
| | Linear | 1,078 | 210 | | 10.3 | |
| | OC | 1,710 | 378 | | 16.3 | |
| 60 sec | SC | 3,585 | 462 | 4921 | 72.8 | 1.6 |
| | Linear | 454 | 210 | | 9.2 | |
| | OC | 882 | 378 | | 17.9 | |

SC: Supercoiled plasmid DNA
Linear: Linear plasmid DNA
OC: Open-Circular plasmid DNA

What is claimed is:

1. An aqueous composition comprising condensed plasmid DNA and a carrier, wherein the carrier comprises a lyophilizable, water-miscible alcohol and a divalent cation.

2. The composition of claim 1, wherein the lyophilizable water-miscible alcohol is tert-butanol.

3. The composition of claim 2, wherein the concentration of tert-butanol is from about 15% to about 35% by volume.

4. The composition of claim 3, wherein the concentration of tert-butanol is from about 17% to about 25% by volume.

5. The composition of claim 4, wherein the concentration of tert-butanol is about 20% by volume.

6. The composition of claim 1, wherein the divalent cation is selected from the group consisting of Ca⁺², Mg⁺² or Zn⁺².

7. The composition of claim 6, wherein the divalent cation is Ca⁺².

8. The composition of claim 7, wherein the concentration of Ca⁺² is from about 0.2 to about 2 millimolar.

9. The composition of claim 8, wherein the concentration of Ca⁺² is about 1 millimolar.

10. The composition of claim 9, wherein the concentration of DNA is from about 10 ug/mL to about 200 ug/mL.

11. The composition of claim 10, wherein the molar ratio of Ca⁺² to DNA-phosphate is about 3.

12. The composition of claim 10, wherein the concentration of Ca⁺² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

13. The composition of claim 1, wherein the counterion to the divalent cation is chloride.

14. The composition of claim 1, wherein the DNA has a negative zeta potential.

15. The composition of claim 1, wherein the DNA is stable to shear stress.

16. The composition of claim 15, wherein the shear stress is sonication-induced.

17. The composition of claim 15, wherein the plasmid DNA remains intact following sonication for 60 seconds using a 50 watt probe sonicator.

18. The composition of claim 17, wherein the total percent of supercoiled, open circular and linear plasmid DNA together after sonication is greater than 90% of its initial value.

19. The composition of claim 1, wherein the DNA in the condensate consists essentially of toroids, rods and spheres.

20. The composition of claim 19, wherein the toroids exhibit a median particle size in the range of from about 50 to about 100 nanometers, as measured by electron microscopy.

21. The composition of claim 1, wherein the condensate exhibits a bimodal particle size distribution.

22. The composition of claim 21, wherein the particle size distribution of the condensate, measured by dynamic light scattering, exhibits peaks in the range of from about 40 to about 70 nanometers and from about 200 to about 500 nanometers.

23. A method to condense plasmid DNA comprising:
(a) preparing an aqueous solution of deionized plasmid DNA;
(b) adding a lyophilizable, water-miscible alcohol to the solution of step (a); and
(c) adding a divalent cation to the mixture of step (b).

24. The method of claim 23, wherein the lyophilizable water-miscible alcohol is tert-butanol.

25. The method of claim 24, wherein the concentration of tert-butanol is from about 15% to about 35% by volume.

26. The method of claim 25, wherein the concentration of tert-butanol is from about 17% to about 25% by volume.

27. The method of claim 26, wherein the concentration of tert-butanol is about 20% by volume.

28. The method of claim 23, wherein the divalent cation is selected from the group consisting of Ca⁺², Mg⁺² or Zn⁺².

29. The method of claim 28, wherein the divalent cation is Ca⁺².

30. The method of claim 29, wherein the concentration of Ca⁺² is from about 0.2 to about 2 millimolar.

31. The method of claim 30, wherein the concentration of Ca⁺² is about 1 millimolar.

32. The method of claim 31, wherein the concentration of DNA is from about 20 ug/mL to about 200 ug/mL.

33. The method of claim 32, wherein the molar ratio of Ca⁺² to DNA-phosphate is about 3.

34. The method of claim 29, wherein the concentration of Ca⁺² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

35. The method of claim 23, wherein the counterion to the divalent cation is chloride.

36. The method of claim 23, wherein the DNA has a negative zeta potential.

37. The method of claim 23, which further comprises removing water and the lyophilizable, water-miscible alcohol from the composition.

38. The method of claim 37, wherein the water and the lyophilizable, water-miscible alcohol are removed by lyophilization.

39. The method of claim 37 which further comprises spray-drying the composition.

40. A composition prepared according to claim 35.

41. A method of protecting DNA against shear stress comprising:
(a) preparing an aqueous solution of deionized plasmid DNA;
(b) adding a lyophilizable, water-miscible alcohol to the solution of step (a); and
(c) adding a divalent cation to the mixture of step (b).

42. The method of claim 41, wherein said shear stress is sonication-induced.

43. The method of claim 41, wherein the lyophilizable water-miscible alcohol is tert-butanol.

44. The method of claim 43, wherein the concentration of tert-butanol is from about 15% to about 35% by volume.

45. The method of claim 44, wherein the concentration of tert-butanol is from about 17% to about 25% by volume.

46. The method of claim 45, wherein the concentration of tert-butanol is about 20% by volume.

47. The method of claim 41, wherein the divalent cation is selected from the group consisting of Ca⁺², Mg⁺² or Zn⁺².

48. The method of claim 47, wherein the divalent cation is Ca⁺².

49. The method of claim 48, wherein the concentration of Ca⁺² is from about 0.2 to about 2 millimolar.

50. The method of claim 47, wherein the concentration of Ca⁺² is about 1 millimolar.

51. The method of claim 50, wherein the concentration of DNA is from about 10 ug/mL to about 200 ug/mL.

52. The method of claim 51, wherein the molar ratio of Ca⁺² to DNA-phosphate is about 3.

53. The method of claim 49, wherein the concentration of Ca⁺² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

54. The method of claim 41, wherein the counterion to the divalent cation is chloride.

55. The method of claim 41, wherein the DNA has a negative zeta-potential.

56. The method of claim 41, which further comprises removing water and the lyophilizable, water-miscible alcohol from the composition.

57. The method of claim 56, wherein the water and the lyophilizable, water-miscible alcohol are removed by lyophilization.

58. The method of claim 55 which further comprises spray-drying the composition.

59. A method for preparing a DNA condensate comprising:
(a) preparing an aqueous solution of deionized plasmid DNA;
(b) adding a lyophilizable, water-miscible alcohol to the solution of step (a); and
(c) adding a divalent cation to the mixture of step (b),
wherein said DNA condensate consists essentially of toroids, rods and spheres.

60. The method of claim 59, wherein the lyophilizable water-miscible alcohol is tert-butanol.

61. The method of claim 60, wherein the concentration of tert-butanol is from about 15% to about 35% by volume.

62. The method of claim 61, wherein the concentration of tert-butanol is from about 17% to about 25% by volume.

63. The method of claim 62, wherein the concentration of tert-butanol is about 20% by volume.

64. The method of claim 59, wherein the divalent cation is selected from the group consisting of Ca⁺², Mg⁺² or Zn⁺².

65. The method of claim 64, wherein the divalent cation is Ca⁺².

66. The method of claim 65, wherein the concentration of Ca⁺² is from about 0.2 to about 2 millimolar.

67. The method of claim 66, wherein the concentration of Ca⁺² is about 1 millimolar.

68. The method of claim 67, wherein the concentration of DNA is from about 20 ug/mL to about 200 ug/mL.

69. The method of claim 68, wherein the molar ratio of Ca⁺² to DNA-phosphate is about 3.

70. The method of claim 66, wherein the concentration of Ca⁺² in millimolar units, is about 16*e^{(0.1386*t)}, wherein t is the volume-percent of tert-butanol, and the counterion to the Ca⁺² is chloride, and wherein the concentration of tert-butanol is from about 15% to about 35% by volume.

71. The method of claim 59, wherein the counterion to the divalent cation is chloride.

72. The method of claim 59, wherein the DNA has a negative zeta potential.

73. The method of claim 59, which further comprises removing water and the lyophilizable, water-miscible alcohol from the composition.

74. The method of claim 73, wherein the water and the lyophilizable, water-miscible alcohol are removed by lyophilization.

75. The method of claim 73 which further comprises spray-drying the composition.

76. The process of claim 59 wherein said toroids, rods and spheres are stable to shear stress.
